(19) United States
(12) Patent Application Publication
(10) Pub. No.: US 2013/0090538 A1
(43) Pub. Date: Apr. 11, 2013

(54) Method and System to Monitor the State and Function of the Cervix and Effects of Treatments for the Cervix

(71) Applicants: Robert Garfield, Friendswood, TX (US); Rainer J. Fink, College Station, TX (US); Jack N. McCrary, Houston, TX (US)

(72) Inventors: Robert Garfield, Friendswood, TX (US); Rainer J. Fink, College Station, TX (US); Jack N. McCrary, Houston, TX (US)

(21) Appl. No.: 13/267,012

(22) Filed: Oct. 6, 2011

(51) Int. Cl. A61B 5/103; A61B 6/00

(52) U.S. Cl. 600/317

(57) ABSTRACT

A method operable to more accurately monitor the state and function of the cervix is provided. Embodiments of the present invention relate generally to detection of fluorescence from the female mammalian cervix. More particularly, embodiments of the present invention relate to predicting true term and preterm labor and delivery, characterizing the function and state of the cervix, and for assessing and monitoring the efficacy of treatments for the cervix. Embodiments of the present disclosure use light induced fluorescence (LIF) via a non-invasive optical probe to accurately and quantitatively measure changes in the cervical tissue during pregnancy and labor. As gestational age increases, the LIF decreases. Ideally, cervical collagen should be at a minimum just prior to delivery. The present invention can be used to monitor the state and function of the cervix during pregnancy, as well as efficacy of cervical treatments during pregnancy.

### TECHNICAL FIELD OF THE INVENTION

Embodiments of the present invention relate generally to detection of fluorescence from the female mammalian cervix. More particularly, embodiments of the present invention relate to predicting true term and preterm labor and delivery, characterizing the function and state of the cervix, and for assessing and monitoring the efficacy of treatments for the cervix.

### BACKGROUND OF THE INVENTION

Spontaneous preterm labor and consequent preterm birth remains as the biggest unsolved obstetrical problem. Approximately 5000 infants die each year in the United States from complications of prematurity and infants born preterm who survive are more likely to develop visual and hearing impairment, chronic lung disease, cerebral palsy, and delayed development in childhood. Although the perinatal mortality rate due to prematurity has decreased dramatically over the past four decades in high-income countries, this reduction has resulted from improvements in neonatal care for premature babies and has occurred in spite of the increasing incidence of premature delivery. Most women who deliver preterm have no apparent risk factors, every pregnancy should therefore be considered to be potentially at risk.

Once preterm labor is established, none of the currently available treatments and interventions can prolong pregnancy sufficiently to allow further intrauterine growth and maturation. The key to treating preterm labor today is its early detection and institution of treatment before any benefit from tocolytic therapy is already lost. This permits delaying delivery long enough to transfer the pregnant patient to the most appropriate hospital, administer corticosteroids and prophylactic antibiotics to reduce neonatal morbidity and mortality.

Accurate early diagnosis of preterm labor is, however, a major problem. Today, up to 50% of patients diagnosed with preterm labor are not actually in preterm labor and as many as 20% of symptomatic patients diagnosed as not being in labor will deliver prematurely. The diagnosis of preterm labor still often relies on presence of contractions.

Fluorescence spectroscopy has been used in applications ranging from cancer screening to drug therapy efficacy. The use of fluorescence spectroscopy dates back to the 1960's when fluorescence spectroscopy was first used for assessing the spin states of nucleic acids (RAHN R O, LONGWORTH J W, EISINGER J, SHULMAN R G. ELECTRON SPIN RESONANCE AND LUMINESCENCE STUDIES OF EXCITED STATES OF NUCLEIC ACIDS. Proc Natl Acad Sci USA. 1964 June; 51:1299-303). Research indicates that collagen measured in cervical tissue may negatively correlated to gestational age (Maul H, Olson G, Fittkow C T, Saade G R, Garfield R E. Cervical light-induced fluorescence in humans decreases throughout gestation and before delivery: Preliminary observations. Am J Obstet Gynecol. 2003 February; 188(2):537-41; Schlembach D, Mackay L, Shi L, Maner W L, Garfield R E, Maul H. Cervical ripening and insufficiency: from biochemical and molecular studies to in vivo clinical examination. Eur J Obstet Gynecol Reprod Biol. 2009 May; 144 Suppl 1:S70-6. Epub 2009 Mar. 20.)

Cervical ripening is a biochemical process where the cervix is conditioned in preparation for delivery of the baby. This conditioning reduces the rigidity and increases the extensibility of the birth canal. Numerous biochemical agents cause an increase in the amount of proteoglycan (decorin) in the cervical tissue. The result is an enzymatic degradation of collagen fibers and a reduction in the concentration of collagen in the cervical tissue that can be measured by optical methods, specifically ultraviolet light induced fluorescence. Sometimes it is desirable to apply pharmaceutical treatments to a patient in order to effect a change in the cervical collagen.

The cervix is composed of two primary components—smooth muscle (˜10%) and connective tissue (˜90%) consisting of collagen, elastin and macromolecular elements that make up the extracellular matrix. Many biochemical and functional changes occur in cervical connective tissue at the end of pregnancy. This process results in softening, dilatation and effacement of the cervix. Ripening is required for appropriate progress of labor and delivery of the baby. The exact mechanisms controlling the cervical ripening process are largely unknown. However, the use of drugs to soften the cervix in order to induce labor and delivery include prostaglandins, oxytocin, antiprogestins, nitric oxide, estrogens and other agents, and the use drugs in order to inhibit ripening to prevent labor and delivery include progestins, antioxytocin agents, atosiban, barusiban, and other agents.

Artificial Reproductive Technology (ART) includes all fertility treatments in which both eggs and sperm are handled. In general, ART procedures involve surgically removing eggs from a woman's ovaries, combining them with sperm in the laboratory, and returning them to the woman's body or donating them to another woman. They do not include treatments in which only sperm are handled (e.g., intrauterine—or artificial—insemination) or procedures in which a woman takes medicine only to stimulate egg production without the intention of having eggs retrieved. It is important to know whether cervical softening agents are effective for these types of procedure (e.g. to test for softening of cervix prior to putting the fertilized ovum into the uterus). There are also gynecologic reasons for which one wants to diagnose the cervix for consistency. There is a need for the ability to monitor the function and state of the cervix in non-pregnant women, as well as the efficacy of cervical treatments in non-pregnant women.

### SUMMARY OF THE INVENTION

Embodiments of the present disclosure are directed to systems and methods that are further described in the following description and claims. Advantages and features of embodiments of the present disclosure may become apparent from the description, accompanying drawings and claims. Embodiments of the present disclosure provide a system or methodology that overcomes the above-noted disadvantages.

In particular, embodiments of the present disclosure provide a system that both overcome the inaccuracy of the toco and the invasive and precarious nature of the IUPC. Embodiments of the present disclosure provide a method operable to more accurately predict true preterm labor and delivery. More specifically, embodiments of the present disclosure provide a method to assess cervical collagen and cervical collagen structures within the cervix. These embodiments at a minimum provide for delivering excitation light to cervical tissue within the cervix. This may be done for example with a probe having both excitation and detection optical fibers. The excitation light causes fluorescence from the cervical tissue wherein the fluorescence is dependent on the cervical collagen and the cervical collagen structures. This fluorescent light is captured again using an optical probe. The fluorescent light captured may then be characterized and a state or function of the cervix may be determined from the characterized fluorescent light.

The light may be delivered and captured using a substantially cylindrical probe that may be covered with a protective sheath having an optical window wherein excitation light passes from and detected fluorescent light passes into the probe from the cervical tissue. The protective sheath may facilitate guiding the cylindrical probe and proper placement. It may also facilitate the insertion and extraction of the probe. A shutter system may couple to a light source to provide the excitation light to the probe wherein the shutter system may limit the exposure of the tissue to the excitation light. Further a signal-processing module may either via analog or digital means collect a dark spectrum and then remove the dark spectrum from the captured fluorescent light to improve the data signal associated with the captured fluorescent light.

Yet another embodiment provides a system that may be used to assess cervical collagen and cervical collagen structures within the cervix. This system includes an excitation light source, an optical probe, a spectroscopy system, and a processor or signal-processing module. The excitation light source delivers excitation light to a probe which in turn delivers the light to cervical tissue within the cervix. The probe also captures fluorescent light from the cervical tissue. The spectroscopy system, optically coupled to the probe, allows the captured fluorescent light to be characterized. This spectroscopy system may optically subtract a dark spectrum from the fluorescent light captured from the cervical tissue or may otherwise digitally subtract the dark spectrum in association with operations by a processor or signal-processing module. The signal-processing module or processor may be used to determine a state or function of the cervix from the captured and characterized fluorescent light. Yet another embodiment of the present disclosure provides a system that may be used to predict true pre-term labor and delivery. This system may include an excitation light source, a shutter system, an optical probe, a spectroscopy system, and a processor or signal-processing module.

Yet another embodiment of the present disclosure by the system operable to predict true preterm labor and delivery. For the reasons stated within the disclosure, it is important to be able to distinguish true pre-term labor and delivery from a false pre-term labor and delivery. Today, there is no accepted method to accurately diagnose true preterm or term labor. The further combination of the EMG based sensing modality (including all possible analysis mentioned above) with analysis of the cervical status using either new instruments such as the SureTouch® collascope, which measures the ripening of the cervix through Light-Induced Auto Fluorescence, or older technologies such as the Bishops Score, or measurement of the cervical length using ultrasound, results in yet a clearer understanding of the status of labor.

### BRIEF DESCRIPTION OF THE DRAWINGS

For a more complete understanding of the present disclosure and the advantages thereof, reference is now made to the following description taken in conjunction with the accompanying drawings in which like reference numerals indicate like features and wherein:

FIG. 1 provides a diagram depicting the Basic elements associated with embodiments of the present disclosure;

FIG. 2 provides a diagram depicting elements, as well as connections and relations of elements associated with embodiments of the present disclosure;

FIG. 3 provides a logic flow diagram for data acquisition in accordance with embodiments of the present disclosure;

FIGS. 4A and 4B are diagrams depicting one possible embodiment of a fiber optic cable bundle in accordance with embodiments of the present disclosure;

FIG. 5 is a diagram depicting one possible embodiment of the probe in accordance with embodiments of the present disclosure;

FIG. 6 provides a data readout showing fluorescent emissions from cervical tissue in accordance with embodiments of the present disclosure;

FIG. 7 provides a summary of cervical fluorescence data acquired from animals during parturition in accordance with embodiments of the present disclosure;

FIGS. 8A, 8B, 8C and 8D provide a summary of cervical fluorescence data from animals treated by various agents to prevent cervical ripening, and using various administration methods, during parturition in accordance with embodiments of the present disclosure;

FIG. 9 provides a summary of cervical fluorescence data from animals treated to prevent induce cervical ripening in accordance with embodiments of the present disclosure; and

FIG. 10 provides a logic flow diagram of a method of predicting true preterm labor and delivery in accordance with embodiments of the present disclosure.

The present disclosure is best understood from the following detailed description when read with the accompanying FIGs., as presented within the text of this application. It is emphasized that, in accordance with the standard practice in the industry, various features are not drawn to scale. In fact, the dimensions of the various features may be arbitrarily increased or reduced for clarity of discussion.

### DETAILED DESCRIPTION OF THE INVENTION

Embodiments of the present disclosure are illustrated in the FIGs., like numerals being used to refer to like and corresponding parts of the various drawings. The following disclosure describes several exemplary embodiments for implementing different features, structures, or functions of the disclosure. Exemplary embodiments of components, arrangements, and configurations are described below to simplify the present disclosure; however, these exemplary embodiments are provided merely as examples and are not intended to limit the scope of the disclosure. Additionally, the present disclosure may repeat reference numerals and/or letters in the various exemplary embodiments and across the FIGs. provided herein. This repetition is for the purpose of simplicity and clarity and does not in itself dictate a relationship between the various exemplary embodiments and/or configurations discussed in the various FIGs. Moreover, the formation of a first feature over or on a second feature in the description that follows may include embodiments in which the first and second features are formed in direct contact, and may also include embodiments in which additional features may be formed interposing the first and second features, such that the first and second features may not be in direct contact. Finally, the exemplary embodiments presented below may be combined in any combination of ways, i.e., any element from one exemplary embodiment may be used in any other exemplary embodiment, without departing from the scope of the disclosure.

Additionally, certain terms are used throughout the following description and claims to refer to particular components. As one skilled in the art will appreciate, various entities may refer to the same component by different names, and as such, the naming convention for the elements described herein is not intended to limit the scope of the disclosure, unless otherwise specifically defined herein. Further, the naming convention used herein is not intended to distinguish between components that differ in name but not function. Further, in the following discussion and in the claims, the terms “including” and “comprising” are used in an open-ended fashion, and thus should be interpreted to mean “including, but not limited to.” All numerical values in this disclosure may be exact or approximate values unless otherwise specifically stated. Accordingly, various embodiments of the disclosure may deviate from the numbers, values, and ranges disclosed herein without departing from the intended scope. Furthermore, as it is used in the claims or specification, the term “or” is intended to encompass both exclusive and inclusive cases, i.e., “A or B” is intended to be synonymous with “at least one of A and B,” unless otherwise expressly specified herein.

In at least one embodiment of the present disclosure, a method for Method and System to Monitor the State and Function of the Cervix and Effects of Treatments for the Cervix is provided. Embodiments of the present disclosure provide a substantially small and light apparatus suitable for research and clinical applications in a hospital, clinic or physician office setting. Embodiments include a display compatible with any laptop computer equipped with proprietary software. Heat released from the light source inside the instrument is minimized through circulation fans. The user interface was also designed to facilitate ease of use. The instrument includes a sterile and/or disposable or reusable and sterilizable sheaths to cover or protect the fiber optic probe during patient examination and to guide the probe, help locate the probe on the tissue properly or in the proper location, and to assist with the insertion or extraction of the probe, during measurement procedures.

Embodiments of the present disclosure are based on fluorescence spectroscopy, a proven and widely-used research tool that can reveal large amounts of information about molecular and physical states. Fluorescent spectra offer important details on the structure and dynamics of macromolecules and their locations at microscopic levels. Fluorescence spectroscopy has been used to examine collagen content for a variety of medical issues, including detection of infection and cancer screening.

As collagen measured in cervical tissue is negatively correlated to gestational age, this relationship results in an ability to quantitatively measure cervical dilation, which, in turn, translates into an accurate methodology of assessing efficacy of medical treatments. Embodiments of the present disclosure may use light induced fluorescence (LIF) via a non-invasive optical probe to accurately and quantitatively measure changes in the cervical tissue during pregnancy and labor. As gestational age increases, the LIF decreases. Ideally, cervical collagen should be at a minimum just prior to delivery. The present invention can be used to monitor the state and function of the cervix during pregnancy, as well as efficacy of cervical treatments during pregnancy.

Embodiments of the present disclosure provide a system and methodology that provides clinicians the ability to monitor efficacy of treatments for cervical conditions, and to assess the state and function of said cervix. These systems and methodologies involve applying a probe to the cervix of a pregnant or non-pregnant female mammal, including humans, with said probe used for illuminating the cervix with excitation light, capturing return fluorescent light emitted from the cervix and making a determination as to the status of the cervix of the patient based on the light induced fluorescence.

One embodiment of the present disclosure provides a system and method to accurately predict true term and preterm labor and delivery in pregnant mammals, including humans. Another embodiment of the present disclosure provides a system and method to accurately assess the function and state of the cervix of pregnant or non-pregnant female mammals, including humans, for the purpose of determining efficacy of cervical treatment from mechanical stimulation, cervical manipulation by hand or device, or chemicals, compounds, pharmaceutical agents, or other treatments (made orally, topically, by injection, or other means of administration). Yet another embodiment of the present invention provides a system and method to aid clinicians in assessing the cervix during gynecologic exams, and during Artificial Reproduction Technology (ART) applications.

A feature of the present disclosure provides a sterilized and/or disposable or reusable and sterilizable cylindrical probe including fiber-optic elements for channeling and delivering excitation light to the cervical tissue, fiber optic elements for standardization of light intensity and fiber optic elements for capturing and channeling return fluorescent light from the cervical tissue.

Another component of the present disclosure provides a sterile cylindrical disposable or reusable and sterilizable sheath for use in covering and protecting the cylindrical probe, with said protective sheath. Said sheath incorporates a window or aperture at one end for allowing passage of light to and from the probe and the cervical tissue regardless whether the light is source or return light.

Another component of the present disclosure provides a sterile and/or disposable or reusable and sterilizable sheath for protecting the probe during measurements or between measurements, for assisting with light collection, for assisting with proper guiding of the probe to the tissue and proper placing of the probe on or near the tissue, and for assisting with insertion or extraction of the probe during measurement procedures.

The present disclosure also may provide a light source for generating excitation light to be delivered to the probe. A fiber optic cable or bundle of fiber optic cables connected at one end to the probe and connected at the other end to the light source, for channeling excitation light from the light source to the probe and for channeling fluorescent light from the probe to the light detector. The light detector may be used for characterizing fluorescent light captured from the cervical tissue.

Another component of the present disclosure provides optical connective junctions, which join together the fiber-optic elements in the probe to the fiber optic cable or cable bundle, and which join together the fiber optic cable or cable bundle to the light detector, and which optimally match in position along a common axis the fiber-optic elements in the probe and the fiber optic cable or cable bundle, and which optimally match in position along a common axis the fiber optic cable or cable bundle and the light detector, with said optical connective junctions engineered in such a way that light transmission through them is maximized, and light reflection and light loss or attenuation in them is minimized. A shutter may be used for controlling the time that the light is transmitted from the light source to the fiber optic cable. This may be accomplished with a foot pedal or other switching mechanism for sending a signal to determine when the shutter will open or close either to collect a dark spectrum or a cervical measurement.

Another component of the present disclosure provides electrical hardware and circuitry for powering, controlling, or regulating the light source, the light detector, the shutter, and the foot pedal signals. Such a processor or signal processing module may be a single processing device or a plurality of processing devices. Such a processing device may be a microprocessor, micro-controller, digital signal processor, microcomputer, central processing unit, field programmable gate array, programmable logic device, state machine, logic circuitry, analog circuitry, digital circuitry, and/or any device that manipulates signals (analog and/or digital) based on operational instructions. The memory (not shown) may be a single memory device or a plurality of memory devices. Such a memory device may be a read-only memory, random access memory, volatile memory, non-volatile memory, static memory, dynamic memory, flash memory, and/or any device that stores digital information. Note that when processing module implements one or more of its functions via a state machine, analog circuitry, digital circuitry, and/or logic circuitry, the memory storing the corresponding operational instructions is embedded with the circuitry comprising the state machine, analog circuitry, digital circuitry, and/or logic circuitry.

Another component of the present disclosure provides computer software or programming code to control the shutter opening and closing, light emission, light detection, quantification of light detected, and saving, analyzing, displaying, and storing of data.

Another embodiment of the present disclosure provides a scoring metric, in which the index generated is related to the fluorescence of the cervix and the physiological state of the cervix and patient and may involve indexes from other instruments, such as uterine electromyography (EMG), cervical ultrasound length, etc., As examples of the scoring metric, the model below could be used to identify patients that need treatments (where device A is uterine EMG and device B is cervical LIF):
i. A (low)+B (high)—use drugs to stimulate contractility (i.e. oxytocin)
ii. A (high)+B (low)—use prostaglandins to stimulate cervical ripening
iii. A (high)+B (low) preterm=use tocolytics
iv. A (high)+B (high or low) preterm=use progesterone to inhibit preterm labor

A second model could also take into account the pushing index (contraction of the abdominal muscles, where pushing is judged on a numerical scale such as a scale of 1 to 10.
i. Where A+B+C=high—likely to deliver vaginally if appropriate
ii. Where A (high)+B (high)+C (low)=unlikely to delivery efficiently
iii. Where A (low)+B (lower)+C (no need to push)

A third model could be used to predict labor based upon measurement of uterine EMG and cervical LIF:
i. A (index of uterine EMG activity—PDS, frequency of bursts, amplitude, etc. on a scale 1 low to 10 high activity)+B (index of Collascope function—collagen content, cervical length or any other index of the cervix on a scale from 1 low, or unripe, to 10, or ripe, cervix).
ii. In patients that are in labor at term or preterm: A+B=Higher values up to 20 means they are in labor
iii. In patients that are not in labor at term or preterm: A+B=Lower values indicate that they are not in labor
iv. Patients with dystocia=Where A is higher (7 to 10) but B is lower (1 to 3)
v. Patients with cervical incompetence=Where A is much lower (1 to 2) and B is much higher (8 to 10).

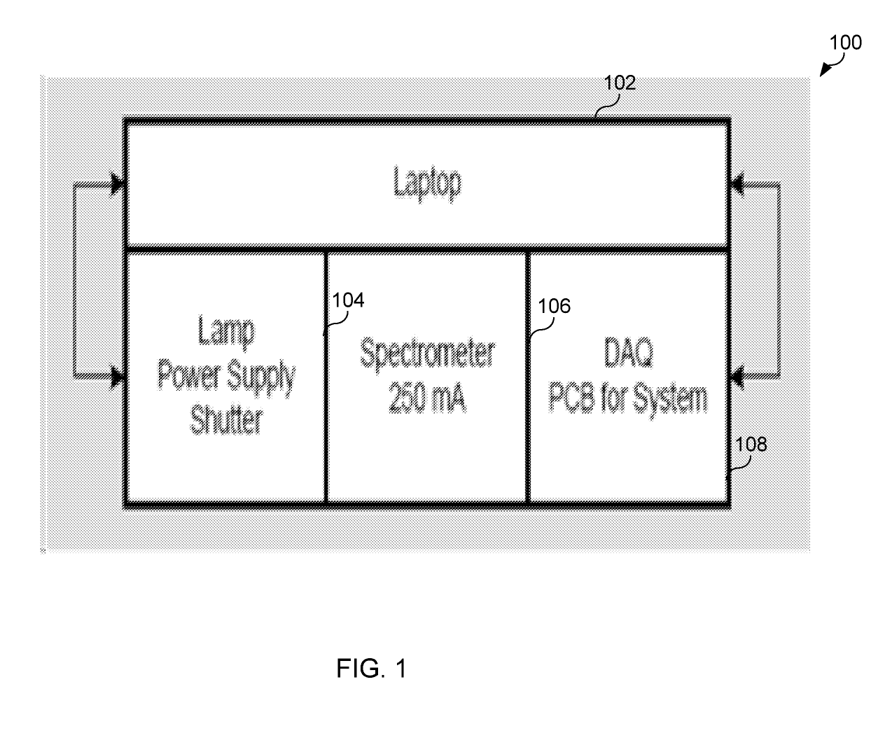

Referring now to FIG. 1, FIG. 1 provides a diagram depicting the Basic elements associated with embodiments of the present disclosure. Block diagram of the system 100 includes a laptop 102 (or other like computing device), lamp with included power supply and shutter 104, a spectrometer or other light acquisition system 106 and a Data acquisition system 108 with included printed circuit board for additional electronic circuitry to be described in subsequent FIGs. Laptop 102 is programmed utilizing software to control functions of all other portions of the disclosed components in FIG. 1.

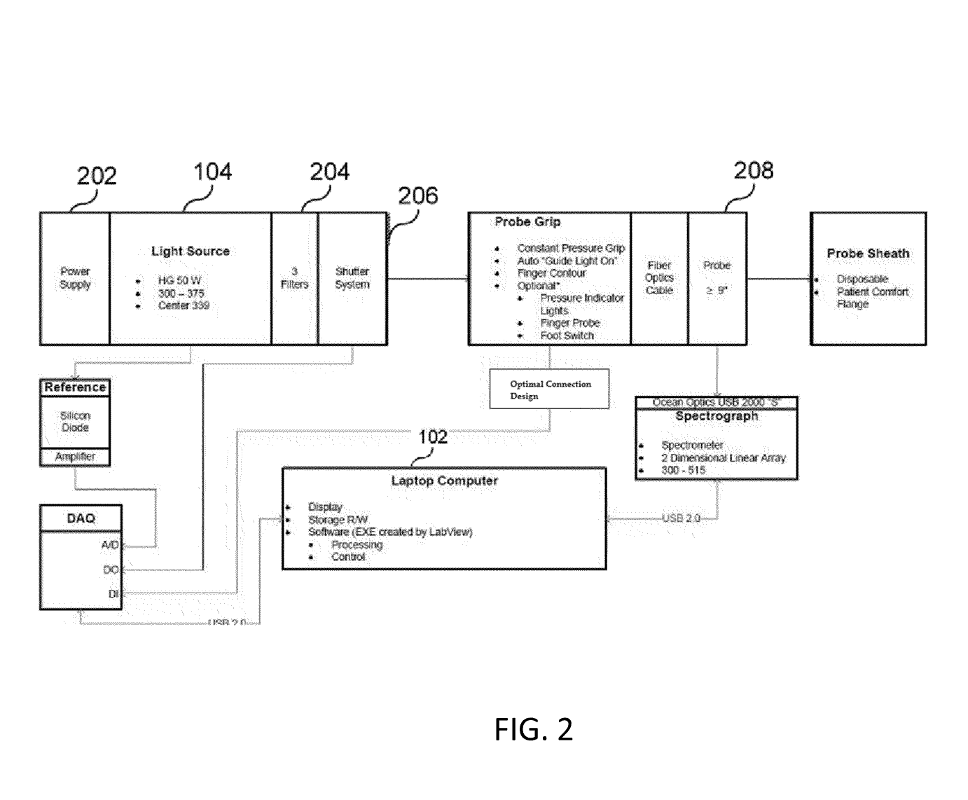

FIG. 2 provides a diagram depicting elements, as well as connections and relations of elements associated with embodiments of the present disclosure. Referring to FIG. 2, Light Source 104 is further disclosed to include a dedicated power supply 202, providing power appropriate for operation of the Light Source 104. The power supply 202 may be a medical grade power supply operable to provide patient isolation and protection. Additionally, light source 104 may preferentially comprise a HG 50 W light source, although other light sources could be anticipated. Current light disclosure includes a light bandwidth between 300 and 375 nm with a center frequency of 339 nm. Light may be further filtered utilizing one or more polarized light filters 204. One embodiment utilizes three polarized light filters. Light application to the surface of the tissue is controlled using a shutter system 206 which allows light to be applied via a probe 208 for a very precise period of time.

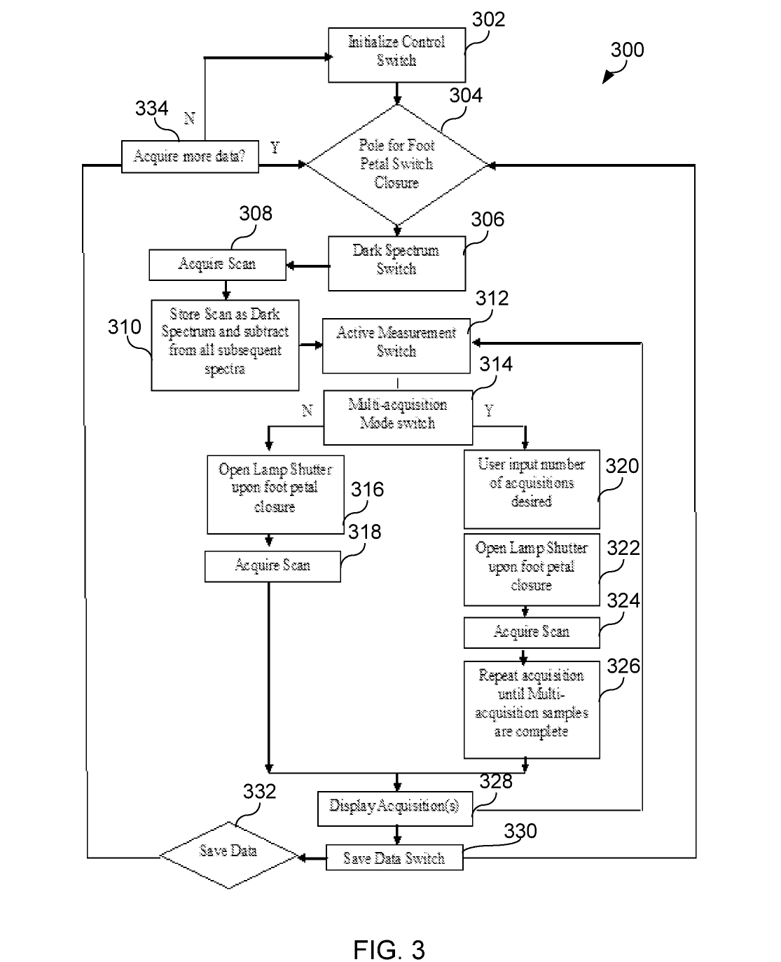

FIG. 3 provides a logic flow diagram for data acquisition in accordance with embodiments of the present disclosure. Referring to FIG. 3, operations 300 are initiated in the Initialize Control Switch block 302. Program flow is continued upon closure of the foot petal switch control signal being recognized by the Data Acquisition system (DAQ) such that the activation of the control switch at decision point 304 indicates that a measurement is being initiated. A dark spectrum switch is activated in block 306 and the spectrum is acquired in block 308 by inserting the probe tip into a dark environment such that the spectrum measured consists of the “dark” or leakage, light inherent in the system. The dark spectrum is stored in block 310. All subsequent spectra have the dark spectrum subtracted mathematically before display such that the displayed data represents only the fluorescent light acquired form the cervix. After acquisition of the dark spectrum, the active measurement control switch is activated in block 312 and a selection of single spectrum or multi spectrum analysis must be made in block 314. In the single spectrum decision branch, a single spectrum will be acquired in block 318 upon the closure of the foot petal switch in block 316. Shutter time can be user controlled by typing the desired shutter open time in a data input block on the graphical user interface. Upon completion of the acquisition of a spectrum, data is displayed to the user in a graphical user interface and is stored on the system hard drive to be retrieved and/or analyzed.

In the multi-acquisition decision branch, shutter time as well as number of acquisitions desired can be entered into a data input block on the graphical user interface in block 320. Individual spectra will be acquired in block 324 on subsequent foot petal control switch activations in block 322, until the user desired number of spectra have been acquired by repeating these steps in block 326. Upon completion of the data acquisition, all data is displayed on the graphical user interface in block 328 and stored at block 332 on the system hard drive or other available memory to be retrieved and or analyzed. User input at block 334 determines if additional spectra are desired. If user input is Yes, the control is returned to the foot petal control switch for reinitiating of the data acquisition process of block 304. If the user input is No, then control is returned to the Initialize control switch of block 302.

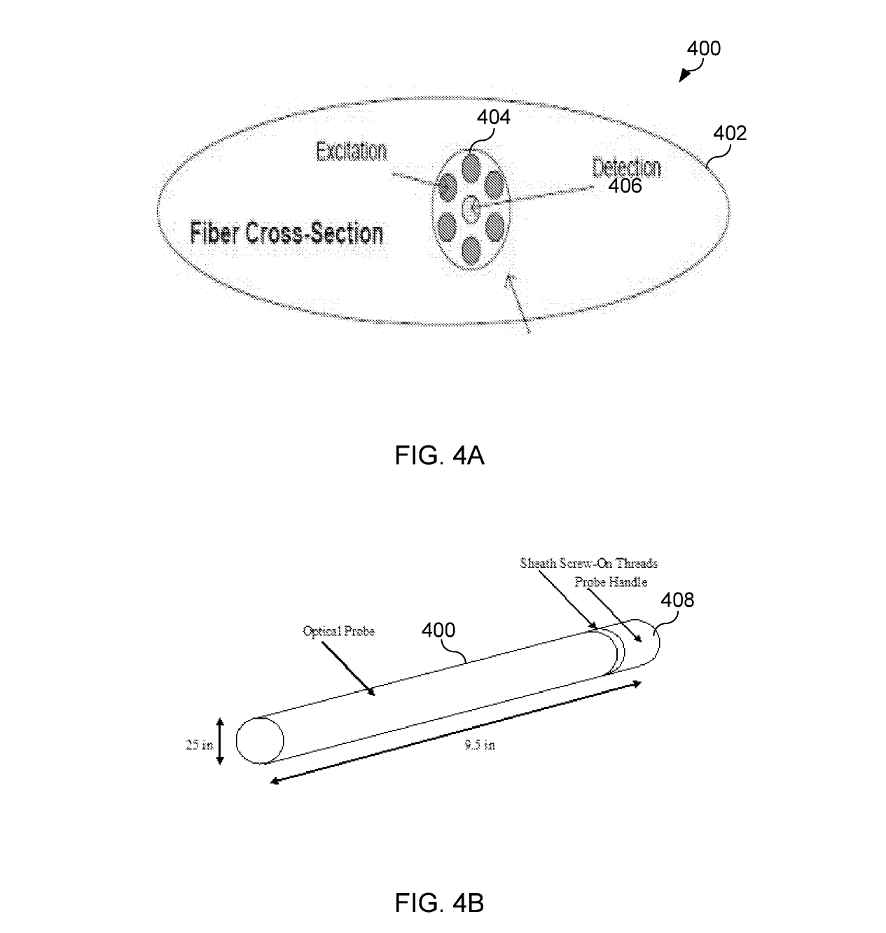

FIGS. 4A and 4B are diagrams depicting one possible embodiment of a fiber optic cable bundle 400 in accordance with embodiments of the present disclosure. Referring to FIG. 4A., a cross section 402 of the fiber optic cable and probe 400 indicates multiple excitation fibers 404 for delivery of incident light from the light source to the cervical tissue—preferably 6 (but could be any number including 1) and detection fibers 406 for return fluorescent light from the cervical tissue to the spectrometer—preferably 1 (but could be multiple). Referring to FIG. 4B, the fiber optic probe 400 may in one embodiment be about 9.5 inches long and has a female thread screw interconnect 408 with the sheath 410 which may be used to cover the probe 400.

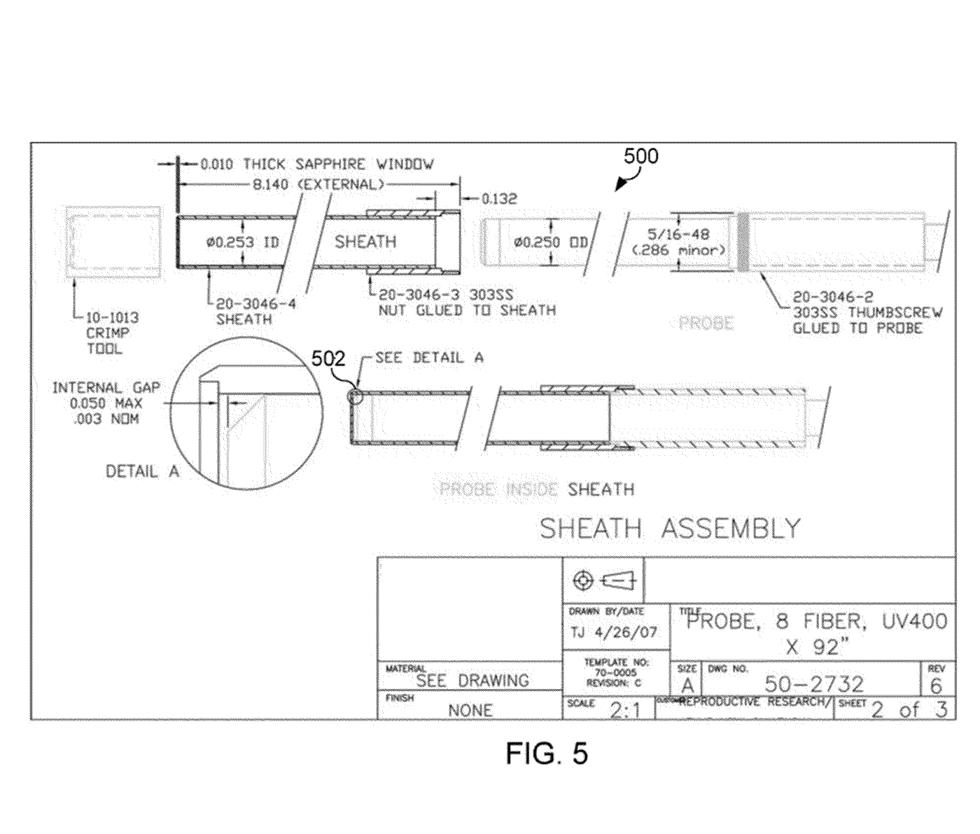

FIG. 5 is a diagram depicting one possible embodiment of the probe in accordance with embodiments of the present disclosure. Referring to FIG. 5, detailed working drawing of the protective sheath 500 which covers the fiber optic probe 400 during a cervical exam. Included in the diagram is one embodiment of the sheath being approximately 8.14″ long with a 0.253″ ID and 0.250″ OD. A sapphire window 502 couples to the patient side of the sheath to allow light to pass without allowing contaminants to soil the fiber optic probe 400. The sheath is attached to the fiber optic probe through a male screw type connection 504 which is crimped on the sheath and then screwed onto female thread 408 of the fiber optic probe 400.

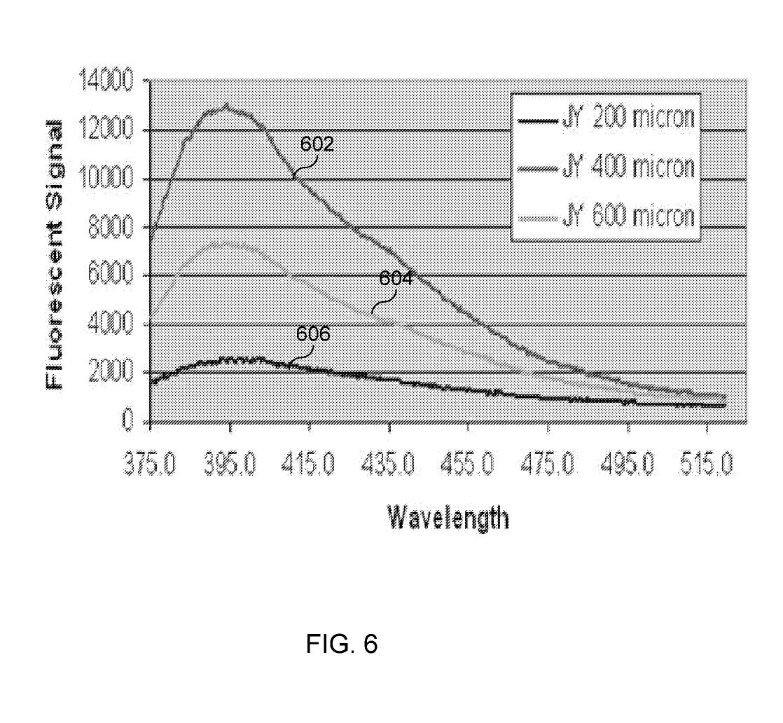

FIG. 6 provides a data readout showing fluorescent emissions from cervical tissue in accordance with embodiments of the present disclosure. Referring to FIG. 6, readout from the embodiments of the disclosure shows fluorescent emissions 602, 604, and 606 from cervical tissue using different sized fiber optic cables to transmit light to and from cervical tissue. As fiber diameter increased, fluorescent signature from collagen in the cervical tissue increased. The signal increase is caused by both an increase in the delivery fiber diameter as well as an increase in the collection fiber diameters.

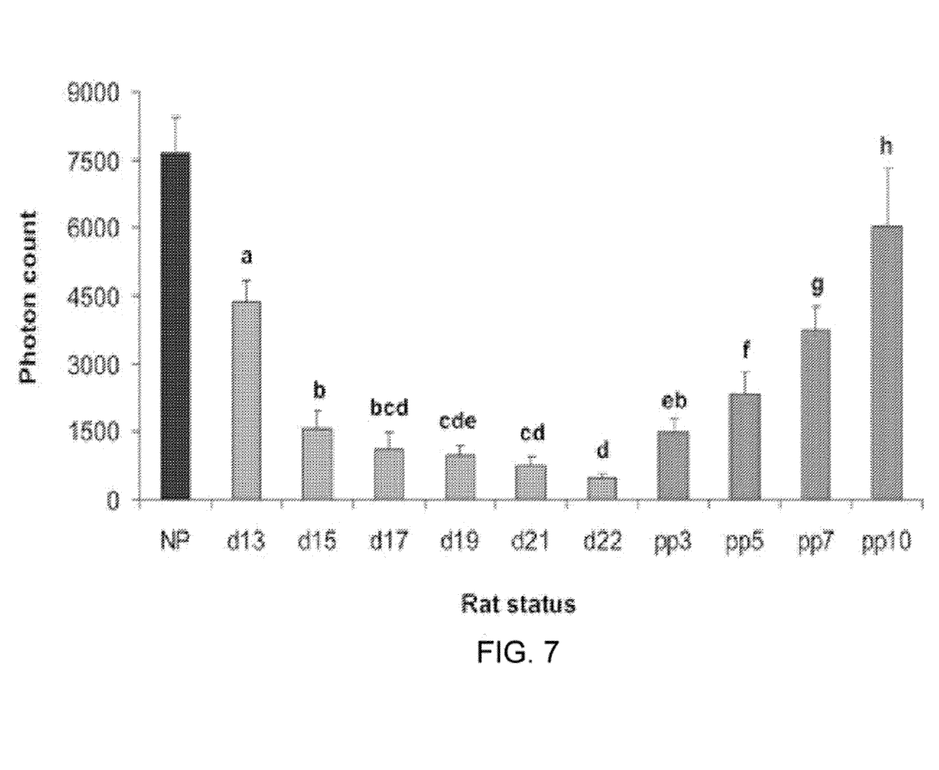

FIG. 7 provides a summary of cervical fluorescence data acquired from animals during parturition in accordance with embodiments of the present disclosure. The amount of cervical collagen was evaluated in vivo by measurement of the auto fluorescent properties of cross-linked collagen. After insertion of a small speculum into the vagina of the anesthetized mammal, the optical probe of the invention was placed on the surface of the exocervix. The probe, which is connected to the main unit of the instrument by a fiber optic cable, delivers not only excitation light (wavelength: 339 nm) onto the cervix but also carries the fluorescent light (mainly caused by pyridinoline cross-links of collagen with a maximum peak at 390 nm) back to the instrument to a spectrometer to display the full spectrum of fluorescence and analyze of the photons emitted by the cervix. In the presented study, the exposure time for excitation was 100 msec. The average of 20 measurements of the detected fluorescent intensity (photon count) at 390 nm was used for each animal at any given time. Measurements of cervical light-induced-fluorescence (LIF) in pregnant, non-treated mammals show a continuously decreasing photon count throughout pregnancy, reaching lowest values at term, and reversal postpartum. After significant (P<0.05) decrease from day 13 to day 15 LIF reaches a wider plateau of non-significant (P>0.05) decreases prior to delivery. LIF values progressively increase postpartum (P<0.05).

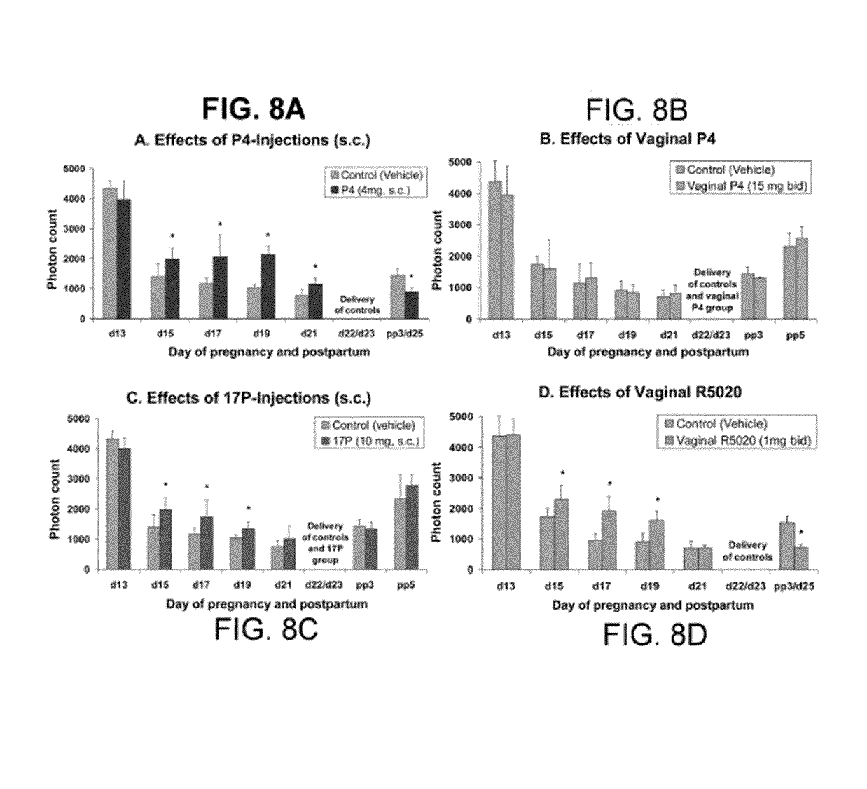

FIGS. 8A, 8B, 8C and 8D provide a summary of cervical fluorescence data from animals treated by various agents to prevent cervical ripening, and using various administration methods, during parturition in accordance with embodiments of the present disclosure. The amount of cervical collagen was evaluated in vivo (only in group s.c. P4, vaginal P4, s.c. 17P, vaginal RS020, s.c. RU-486) by measurement of the auto fluorescent properties of cross-linked collagen. After insertion of a small speculum into the vagina of the anesthetized mammal, the optical probe of the invention was placed on the surface of the exocervix. The probe, which is connected to the main unit of the instrument by a fiber optic cable, delivers not only excitation light (wavelength: 339 nm) onto the cervix but also carries the fluorescent light (mainly caused by pyridinoline cross-links of collagen with a maximum peak at 390 nm) back to the instrument to a spectrometer to display the full spectrum of fluorescence and analyze of the photons emitted by the cervix. In the current study, the exposure time for excitation was 100 msec. The average of 20 measurements of the detected fluorescent intensity (photon count) at 390 nm was used for each patient at any given time. Measurements of cervical light-induced fluorescence (LIF) were performed on non-pregnant mammals once and in pregnant mammals every other day starting at day 13 of gestation until delivery and on postpartum day 3 and/or postpartum day 5. LIF is significantly (P<0.05) higher in the P4 injection group compared with vehicle controls for any day of gestation (FIG. 8A). There are no significant differences (P>0.05) between the vaginal P4 group and vehicle controls at any time in gestation (FIG. 8B). LIF is significantly higher (P<0.05) in the 17P treated group (until day 19 only) compared with vehicle controls (FIG. 8C). LIF is significantly higher (P<0.05) in the RS020 treated group (until day 19 only) compared with vehicle controls (FIG. 8D).

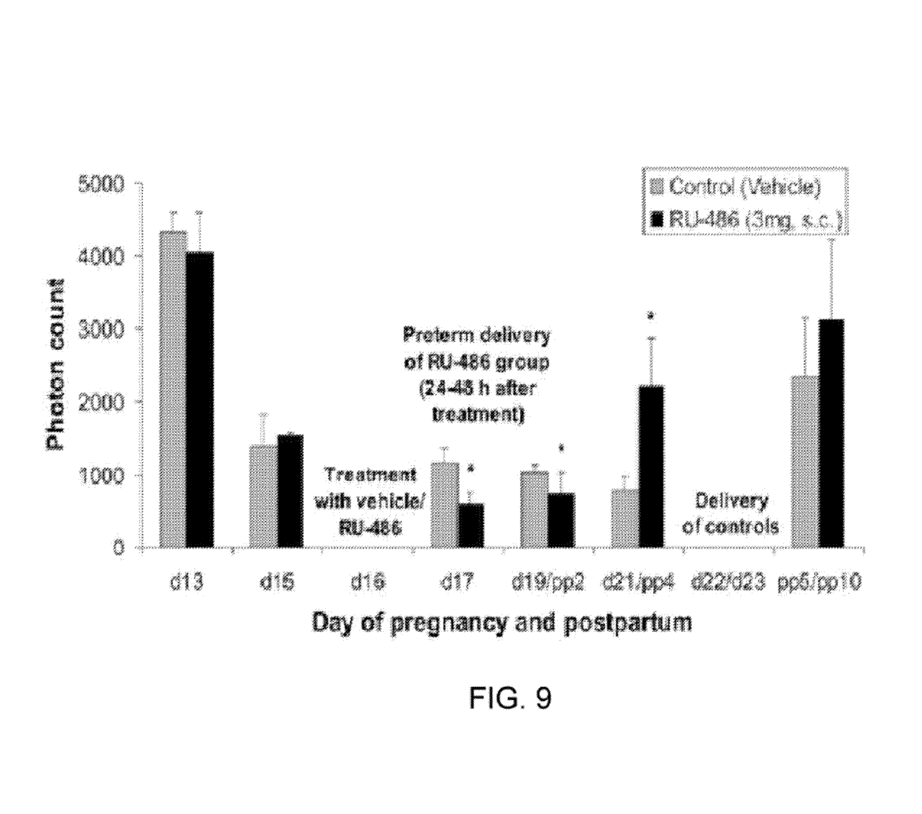

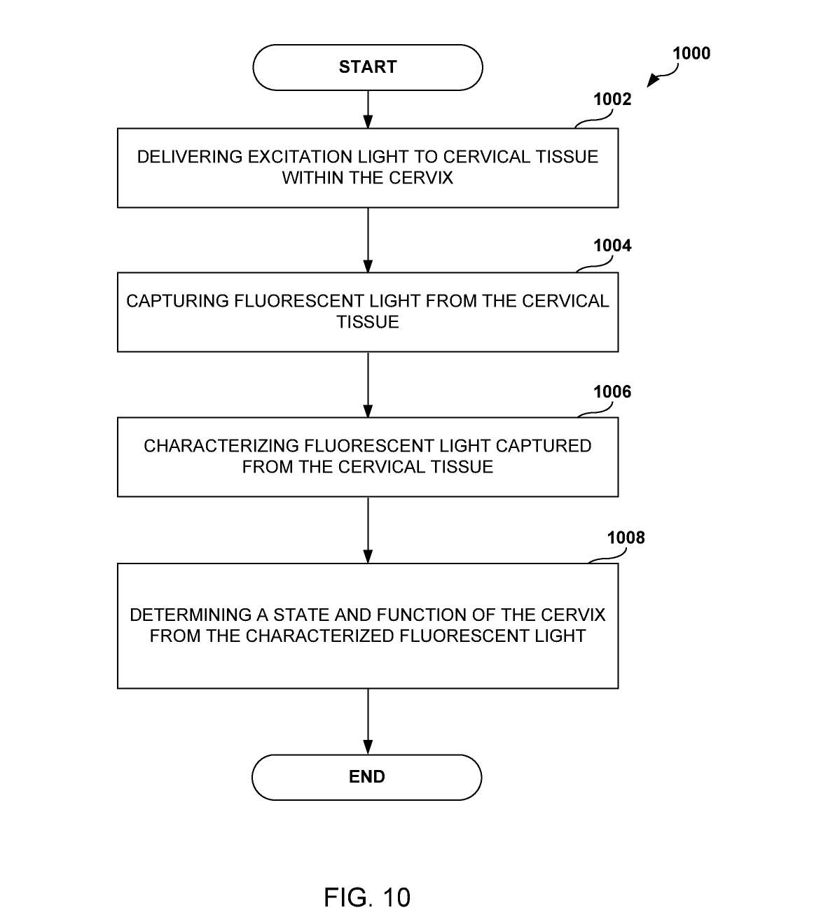

FIG. 9 provides a summary of cervical fluorescence data from animals treated to prevent/induce cervical ripening in accordance with embodiments of the present disclosure. The amount of cervical collagen was evaluated in vivo by measurement of the auto fluorescent properties of cross-linked collagen. After insertion of a small speculum into the vagina of the anesthetized mammal, the optical probe of the invention was placed on the surface of the exocervix. The probe, which is connected to the main unit of the instrument by a fiber optic cable, delivers not only excitation light (wavelength: 339 nm) onto the cervix but also carries the fluorescent light (mainly caused by pyridinoline cross-links of collagen with a maximum peak at 390 nm) back to the instrument to a spectrometer to display the full spectrum of fluorescence and analyze of the photons emitted by the cervix. In the current study, the exposure time for excitation was 100 msec. The average of 20 measurements of the detected fluorescent intensity (photon count) at 390 nm was used for each animal at any given time. LIF before treatment at day 13 shows no significant differences (P>0.05) between the treatment and the control group. LIF is significantly lower (P<0.05) in the RU-486 treated group 24 and 72 hours after treatment compared with vehicle controls. LIF is higher in the RU-486 treated group 5 days after treatment compared with vehicle controls (P<0.05). FIG. 10 provides a logic flow diagram of a method of predicting true preterm labor and delivery in accordance with embodiments of the present disclosure. This may be accomplished by assessing cervical collagen and cervical collagen structures within the cervix. Operations 1000 begin with Block 1002 where excitation light is delivered to cervical tissue within the cervix. This may be done using a cervical probe wherein an optical fiber within the probe is used to deliver light to the tissue of the cervix. This light induces LIF. The optical probe then also captures fluorescent light from the cervical tissue in Block 1004. It should be noted that the excitation light may be controlled using a shutter system or other like system to limit the excitation light that the cervical tissue is exposed to.

In Block 1006 a spectroscopy system may be used to characterize the fluorescent light captured from the cervical tissue. Then in Block 1008 a state and function of the cervix may be determined from the characterized fluorescent light. In optional embodiments of the present invention a dark spectrum may be taken prior to the capture of the fluorescent light from the cervix. This dark spectrum, via analog or digital methods, may be subtracted from the spectrum associated with the captured fluorescent light in order to improve the quality of the data signal associated with the captured fluorescent light.

In summary, embodiments of the present disclosure provide a method operable to more accurately monitor the state and function of the cervix. Embodiments of the present disclosure relate generally to detection of fluorescence from the female mammalian cervix for predicting true term and preterm labor and delivery, characterizing the function and state of the cervix, and for assessing and monitoring the efficacy of treatments for the cervix. LIF is induced and measured via a non-invasive optical probe to accurately and quantitatively measure changes in the cervical tissue during pregnancy and labor. As gestational age increases, the LIF decreases; thus, embodiments can be used to monitor the state and function of the cervix during pregnancy, as well as efficacy of cervical treatments during pregnancy.

The foregoing has outlined features of several embodiments so that those skilled in the art may better understand the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions and alterations herein without departing from the spirit and scope of the present disclosure.

What is claimed is:

1. A method operable to assess cervical collagen and cervical collagen structures present in a cervix of a mammal comprising:
delivering excitation light to cervical tissue within the cervix;
capturing fluorescent light from the cervical tissue;
characterizing fluorescent light captured from the cervical tissue; and
determining a state and function of the cervix from the characterized fluorescent light.

2. The method of claim 1, wherein a cylindrical probe delivers excitation light to cervical tissue within the cervix, and captures fluorescent light from the cervical tissue.

3. The method of claim 2, wherein the cylindrical probe is covered with a protective sheath, the sheath comprising an optical window operable to pass light to and from the probe and the cervical tissue.

4. The method of claim 2, wherein the cylindrical probe comprises at least one excitation fiber and at least one detection optical fiber.

5. The method of claim 3, wherein the protective sheath is operable to:
facilitate guiding of the cylindrical probe to the cervical tissue;
proper placement of the cylindrical probe proximate to the cervical tissue; and
facilitate insertion and/or extraction of the cylindrical probe.

6. The method of claim 2, wherein a light source operable to generate the excitation light optically couples to the cylindrical probe and delivers the excitation light to the cylindrical probe.

7. The method of claim 2, wherein a light detector operable to characterize fluorescent light captured from the cervical tissue optically couples to the cylindrical probe.

8. The method of claim 2, wherein a shutter system controls a time that the excitation light is delivered to the cervical tissue within the cervix.

9. The method of claim 1, further comprising:
collecting a dark spectrum; and
subtracting the dark spectrum from the captured fluorescent light.

10. A system operable to assess cervical collagen and cervical collagen structures present in a cervix of a mammal comprising:
an excitation light source;
a probe optically coupled to the excitation light source, the probe operable to:
deliver excitation light to cervical tissue within the cervix; and
capture fluorescent light from the cervical tissue;
a spectroscopy system optically coupled to the probe, the spectroscopy system operable to characterize fluorescent light captured from the cervical tissue; and
a processor operable to determine a state and function of the cervix from the characterized fluorescent light.

11. The system of claim 10, wherein the probe further comprises: a first set of optical fibers operable to deliver the excitation light to cervical tissue within the cervix; and a second set of optical fibers operable to capture fluorescent light from the cervical tissue.

12. The system of claim 10 further comprising a protective sheath operable to couple to the probe, the protective sheath comprising an optical window operable to pass light to and from the probe and the cervical tissue.

13. The system of claim 10, wherein the protective sheath is operable to:
facilitate guiding of the cylindrical probe to the cervical tissue;
proper placement of the cylindrical probe proximate to the cervical tissue; and
facilitate insertion and/or extraction of the cylindrical probe.

14. The system of claim 10, wherein the probe comprises at least one excitation fiber and at least one detection optical fiber.

15. The system of claim 10, further comprising a shutter system operable to control a time that the excitation light is delivered to the cervical tissue within the cervix.

16. The system of claim 10, wherein the system is operable to collect a dark spectrum, and the processor and/or spectroscopy system is operable to subtract the dark spectrum from the captured fluorescent light.

17. A system operable to assess cervical collagen and cervical collagen structures present in a cervix of a mammal comprising:
an excitation light source;
a shutter system optically coupled to the excitation light source, the shutter system operable to control a time that the excitation light is delivered to the cervical tissue within the cervix
a probe optically coupled to the excitation light source and shutter system, the probe further comprises:
a first set of optical fibers operable to deliver the excitation light to cervical tissue within the cervix; and
a second set of optical fibers operable to capture fluorescent light from the cervical tissue.
the probe operable to:
deliver excitation light to cervical tissue within the cervix; and
capture fluorescent light from the cervical tissue;
a protective sheath operable to couple to the probe, the protective sheath comprising an optical window operable to pass light to and from the probe and the cervical tissue;
a spectroscopy system optically coupled to the probe, the spectroscopy system operable to characterize fluorescent light captured from the cervical tissue; and
a processor operable to determine a state and function of the cervix from the characterized fluorescent light.

18. The system of claim 17, wherein the protective sheath is operable to:
facilitate guiding of the cylindrical probe to the cervical tissue;
proper placement of the cylindrical probe proximate to the cervical tissue; and
facilitate insertion and/or extraction of the cylindrical probe.

19. The system of claim 17, wherein the probe comprises at least one excitation fiber and at least one detection optical fiber.

20. The system of claim 17, wherein the system is operable to collect a dark spectrum, and the processor and/or spectroscopy system is operable to subtract the dark spectrum from the captured fluorescent light.

21. The system of claim 17, wherein the processor compares a spectrum of the captured fluorescent light to a labor predictive spectrum,
wherein a favorable comparison between the labor predictive spectrum and the captured spectrum indicates an increased probability of True Preterm Labor and Delivery.

22. The System of claim 21, wherein an increased probability of True Preterm Labor and Delivery results in delivery within a predefined number of days.

23. A system operable to predict True Preterm Labor and Delivery, the system comprising:
an excitation light source;
a shutter system optically coupled to the excitation light source, the shutter system operable to control a time that the excitation light is delivered to the cervical tissue within the cervix
a probe optically coupled to the excitation light source and shutter system, the probe further comprises:
a first set of optical fibers operable to deliver the excitation light to cervical tissue within the cervix; and
a second set of optical fibers operable to capture fluorescent light from the cervical tissue.
the probe operable to:
deliver excitation light to cervical tissue within the cervix; and
capture fluorescent light from the cervical tissue;
a protective sheath operable to couple to the probe, the protective sheath comprising an optical window operable to pass light to and from the probe and the cervical tissue;
a spectroscopy system optically coupled to the probe, the spectroscopy system operable to characterize fluorescent light captured from the cervical tissue; and
a signal processing module communicably coupled to the spectroscopy system, the signal processing module operable to:
determine a state and function of the cervix from the characterized fluorescent light;
compare a captured spectrum to a labor positive predictive spectrum, wherein a favorable comparison between the captured spectrum to a labor positive predictive spectrum indicates an increased probability of True Preterm Labor and Delivery; and
display a communication indicating the increased probability of True Preterm Labor and Delivery.
